(19) United States
(12) Patent Application Publication
(10) Pub. No.: US 2021/0113441 A1
(43) Pub. Date: Apr. 22, 2021

(54) BIOSOLVENT MIXTURE REMOVAL OF INFECTION-CAUSED NAIL DISCOLORATION

(71) Applicant: Vertec BioSolvents, Inc., Downers Grove, IL (US)

(72) Inventors: James E. Opre, Downers Grove, IL (US); Rathin Datta, Chicago, IL (US)

(73) Assignee: Vertec BioSolvents, Inc., Downers Grove, IL (US)

(21) Appl. No.: 16/781,845

(22) Filed: Feb. 4, 2020

Related U.S. Application Data
(60) Provisional application No. 62/916,552, filed on Oct. 17, 2019.

(51) Int. Cl. A61K 8/34; A61K 8/37; A61K 47/10; A61K 47/14; A61K 31/4174; A61K 31/69; A61Q 3/00

(52) U.S. Cl. CPC A61K 8/34; A61K 8/37; A61K 47/10; A61K 47/14; A61K 8/31; A61K 31/69; A61Q 3/00; A61K 2800/48; A61K 2800/31; A61K 31/4174

(57) ABSTRACT

A method for removing infection-caused nail discoloration is disclosed that contemplates the steps of a) contacting a fingernail and/or toenail in need removal of infection-caused nail discoloration with a composition containing a mixture of two major components, A and B. Component A is a C₂-C₄ alcohol present at about 90% to about 20% (w/w), and Component B is a lactate ester of the same or different C₂-C₄ alcohol(s) present at about 10% to about 80% (w/w). A contemplated composition optionally contains up to about 5% (w/w) each of one or more of a monoterpene or monoterpene mixture, thickening agent and C₁-C₄ alcohol C₁₂-C₂₀ carboxylate. That contact is maintained for a time period sufficient for the composition to be sorbed by the nail and/or evaporate, step b). Steps a) and b) are repeated about 5 to about 14 times per week until the nail discoloration is no longer observable.

### CROSS-REFERENCE TO RELATED APPLICATIONS

This application claims priority from application Ser. No. 62/916,552 that was filed on Oct. 17, 2019, whose disclosures are incorporated herein by reference.

### BACKGROUND ART

Discolorations from biological infections are very prevalent in many people, particularly those that suffer or have suffered from chronic fungal infections. These discolorations are often brownish or reddish in color and unsightly. These are often embedded inside the nail or in between the skin and nail or in both areas. Often there is other biological debris associated with these discolorations.

The discolorations are extremely difficult to remove. Very often they last for years even after the infection is eliminated or abated by medicines or bioactive agents. Even though a new nail growing from the base is clear these stains stay on and are not removed by the new nails' natural growth. This has been a major problem and millions of people suffer from it.

There are many commercial products as well as treatments by prescription medicines for toe and nail fungus. Clotrimazole, an antifungal agent, is a very well-known medicine that is claimed to eliminate or abate “athlete's foot” and toe fungus. Popular brand name products such as Lotrimin® and other non-branded products use clotrimazole as the active agent in various formulations such as ointments and lotions.

Itraconazole, another azole antifungal active ingredient, is used in another brand named product Sporanox®. Efinaconazole is the active agent used in a brand named product Jublia®.

Prescription medicines are sometimes given. Terbinafine hydrochloride is the anti-fungal medication in the brand named product Lamisil®. A benzoxaborole compound, tavaborole, is the anti-fungal medication in Keridyn® which is used in special cases. Tavaborole and similar benzoxaborole compounds that are free of ionically charged substituent groups at physiologic pH values are themselves free of ionic charge at pH 7.2-7.4.

In addition, many of the azole-type antifungals have a positive charge in aqueous environments and it is preferred that an antifungal compound be free of ionic charge at physiological pH values of about 7.2-7.4. Thus, a benzoxaborole compound such as tavaborole that is uncharged at physiological pH values is preferred in some embodiments.

Most of the prescription products that are ingested for their bioactivity have side effects such as gastrointestinal discomfort, liver inflammation and worsening of existing heart failure. They can often interfere with the metabolism of other medications.

Many “natural,” “holistic” treatments are often promoted and endorsed. Essential oils, particularly tea tree oil, have been claimed to be effective in toe fungus treatment. Others promote bolstering the immune system by taking probiotics—strains of lactobacilli, turmeric and other herbs. Still others promote oxidizing cleaning agents such as hydrogen peroxide, chlorine, whereas others promote known antibacterials and surfactants.

None of the above products and treatments actually eliminate the discolorations. Furthermore, their effectiveness, if any, is diluted out or removed when they get diluted by water either from the body or by bathing.

Recently, laser treatments have become popular and more common. Often they are successful in treating the infection but are not effective in clearing the discolorations.

As is disclosed hereinafter, a composition used in a contemplated method surprisingly does remove finger and toe nail discoloration.

### SUMMARY OF THE INVENTION

The invention contemplated here is a method for removal of stains, discolorations and biological debris caused by infection of nails (infection-caused nail discoloration), using a mixture of biosolvents. Use of this method leads the user to clear and healthy-looking nails that last for a long period of time.

A contemplated method for removing infection-caused nail discoloration utilizes the steps of a) contacting a fingernail and/or toenail in need of removal of infection-caused nail discoloration with a composition containing a mixture of two major components, A and B. Component A is a C₂-C₄ alcohol that is present at about 90% to about 20% (w/w), and Component B is a lactate ester of the same or different C₂-C₄ alcohol(s) that is present at about 10% to about 80% (w/w). A contemplated composition optionally contains up to about 5% (w/w) each of one or more of (i) a monoterpene or monoterpene mixture, (ii) a thickening agent and (iii) a C₁-C₄ alcohol C₁₂-C₂₀ carboxylate. That step b) contact is maintained in the absence of added water contact for a time period sufficient for the composition to be sorbed by the nail and/or evaporate. Steps a) and b) are repeated at least 5 to about 7 times per week until the nail discoloration is no longer observable.

A pharmaceutical composition for treating a dermatological fungal infection is also contemplated. Such a composition contains a therapeutic amount of an azole, benzoxaborole, other anti-fungal compounds that are commonly used as an active ingredient in commercial medicament treatments for nail infections or mixtures thereof dissolved or dispersed in a contemplated biosolvent mixture. The resulting composition can be applied to a fingernail or toenail as discussed above to treat a fungal infection while also being used to remove infection-caused nail discoloration.

Furthermore, the method is very easy to use. It only requires topical application to the affected area by a swab a few times per day.

### BRIEF DESCRIPTION OF THE DRAWINGS

In the drawings forming a portion of this disclosure and are more fully discussed in the following Example of the same numeral as a Figure:

FIG. 1A is an enlarged photograph of a dorsal view of the toes of a 69-year old female subject taken approximately 3 weeks after beginning application to the right hallux nail using a composition and method of the invention of (Example 1; Formulation 1) and to the left hallux nail with Listerine®. The two toenails began the treatments with similar discolorations from a foot fungus infection. FIG. 1B is a photograph of the same female subject's toes after another two weeks of applications and shows a color improvement to the right toe treated according to this invention and no change in the left toe nail treated with Listerine®. FIG. 1C is another photo of the same subject's toes taken approximately 16 weeks after the beginning of the applications to the right toe began. In the meantime, treatment of the left toe began following the regimen of the right toe's treatment beginning about 8 weeks prior to the photograph of FIG. 1C being taken. As is seen, the growth of the right toe nail has continued growing and remained completely clear and healthy looking. The left toe exhibited slow and continuing growth of clear growth. FIG. 1D is a photograph taken about 8 weeks after treatment began on the nail of the left hallux and about 16 weeks after treatment began on the nail of the right halux. FIG. 1E shows results after 32 weeks of application to the right hallux and 16 weeks of application to the left hallux. FIG. 1F is a photograph taken about 12 weeks later.

FIG. 2 is a photograph that shows the left foot of a 65 year old male subject who had had a chronic toenail fungal infection that had persisted over decades, primarily on his left foot. After extensive physical and topical treatments to the toes of his left foot including removal nails of the hallux, fourth and fifth toes. The left hallux toenail, which was discolored for a long time period, was treated once daily (with about 90-95% use consistency) using a method of the invention. About 12 weeks after beginning of treatment, the photo of FIG. 2 was taken and shows apparent clear new growth extending from the lunule into the nail body.

FIG. 3 is a photograph of the feet of a 73 year old male volunteer who had chronic history of fungal toe infection and nail discoloration. This subject received treatment using Ciclopirox Olamine Cream USP, 0.77% on the nails of the hallux and second toe of his right foot that proved to be ineffective. Those two toenails, only, were treated by once daily application with a composition as above. The photograph of FIG. 3 was taken about six months after beginning of the treatment of the two right toenails with a composition and method described herein. The two toes show that the old growth of the two toenails was stable and new, clear growth was slowly becoming apparent.

FIG. 4 is a photograph of the feet of Subject 4 who is a 78 year old male who has suffered from a persistent toe fungus problem and discoloration for the last 30 years after twice daily applications of two formulations of this invention. The subject applied VertecBio solvent ToeMed™ Formulation #1 twice a day using a cotton swab for a period of approximately 20 weeks. Signs of clearing of the discolorations began after about 12 weeks. He switched to VertecBio solvent ToeKleer™ Formulation #2 using the same application method for an additional 12 weeks.

FIG. 5A-5E are photographs of the feet of a seventy-six-year-old man (Subject 5) before and during a study of the effects of application of a formulation of this invention. The subject began application once daily of Biosolvent Formulation 1 described earlier to all of the nails of both feet. Photos of the subject's feet and toenails were taken prior to the beginning of the study (FIG. 5A) and approximately monthly thereafter (FIGS. 5B-5E). After about four weeks beginning applications, the lunules of the hallux toenail showed signs of clarity and normal growth (FIG. 5B). After about another 3 weeks, the clarity and normal growth appears to have progressed from the lunules into the nail body with the hallux nails having about a quarter inch of new growth (FIG. 5C). About seven weeks after the start of any biosolvent formulation application to the subject's toenails, the subject switched to administration of Biosolvent Formulation 2 that is also described previously herein. The first photo after the change to administration of Biosolvent Formulation 2 was taken about six weeks after the change to Formulation 2 was made (FIG. 5D). The photo of FIG. 5E, taken about one month after that of FIG. 5D, shows that substantially all of the brown-colored material beneath the toenails has gone and has not been replaced.

### DETAILED DESCRIPTION OF PREFERRED EMBODIMENTS

The present invention contemplates a method for removing infection-caused nail discoloration. A contemplated method utilizes the steps of a) contacting a fingernail and/or toenail in need of removing infection-caused nail discoloration with a composition comprising a mixture of two major components, A and B. Component A is a C₂-C₄ alcohol that is present at about 90% to about 20% (w/w), and Component B is a lactate ester of the same or different C₂-C₄ alcohol(s) that is present at about 10% to about 80% (w/w). The composition optionally contains up to about 5% (w/w) each of one or more of (i) a monoterpene or a monoterpene mixture, (ii) a thickening agent and (iii) a C₁-C₄ alcohol C₁₂-C₂₀ carboxylate. The b) contact is maintained in the absence of water contact (added water; e.g., rinsing or remnants of bath or shower) for a time period sufficient for the composition to be sorbed by the nail and/or evaporate. Steps a) and b) are repeated at least 5 to about 7 times per week until the nail discoloration is no longer observable. Those steps can also be continued indefinitely, or until the nails receiving the application grow out to the extent desired by the user.

The word “sorbed” is used herein to mean “taken up” without preference for one or the other of absorbed or adsorbed. The word “observable” is used to mean visible to the treated subject without aid of magnification other than that provided by regular eye wear and from a distance of the subject eyes to feet when standing.

Application of a biosolvent to contact the nail is preferably carried out not only on the nail surface itself, but also to contact the lateral and proximal nail folds and free edge that surround a contacted nail. The lateral and proximal nail folds are often referred to as the cuticle, but are believed to more correctly be referred to as stated here.

That contacting of a nail with a contemplated biosolvent is carried out at least about once per day, or about 5 to about 7 times each week, and preferably at least daily (7 times). Although there is not a known upper limit to the number of times the contacting can be carried out, it has been found to be convenient and effective for one application each morning and another application before retiring to sleep. Thus, applications are preferably carried out about twice daily or about 10 to about 14 times per week, allowing for some missed treatments. More broadly, a toenail or fingernail is contacted with a contemplated biosolvent about 5 to about 14 times per week (7 days).

In examining the two major components of a contemplated biosolvent, it is preferred that the C₂-C₄ alcohol of Component A be a single alcohol, and that the C₂-C₄ alcohol of the lactate ester of Component B also be a single alcohol, and more preferably be the same alcohol as that of Component A. It is particularly preferred that Component A be ethanol and that Component B be ethyl lactate.

A preferred concentration is about 10% to about 80% (w/w) of the ester Component B, with a more preferred amount being about 15% to about 40% (w/w), with remainder as the alcohol of Component A. Small quantities [each about 0.1 to about 5% (w/w)] of additives such as (i) a monoterpene or monoterpene mixture, (ii) an emollient/penetrant that aids in transport, and (iii) a thickener (viscosifier) can also be present so that the sum of the components totals 100% with any deficit being made up by one or both of the alcohol and lactate ester portions.

It is preferred that water not be added to a contemplated composition. Stated differently, preferably, a contemplated composition is free of added water. Thus, water is not consciously added to a contemplated biosolvent mixture prior to its being used.

It is to be understood that either or both of Components A and B are miscible with water and some are hygroscopic so that up to about 5% (w/w) of water can be present as an impurity that can be due to air-borne humidity, incomplete separation during distillation, water on a finger or toenail surface after cleaning, such as after bathing, and not dried off. As is well known, ethanol and water form an azeotropic mixture that contains 95.6% ethanol with the rest being water.

In U.S. Pat. No. 9,963,660 whose contents are incorporated by reference, it was disclosed that a biosolvent mixture such as that utilized in a contemplated method could dissolve food or bioprocess-derived contaminants such as carbohydrates, proteins and fats from surfaces used in food processing and preparation of meat and vegetables (typically hard, impervious surfaces) in presence of some water and provide bacteriostatic activity for a period of time even after water dilution. The individual solvents did not provide these enhanced activities.

This prior work, which was focused on cleaning hard and/or non-porous surfaces of food and other processing equipment and machinery, did not remotely suggest that such solvent mixtures would be useful on active biological surfaces and not only remove and clean the debris and discolorations but also clear them and the new growth would remain clear and cure this problem for a long period of time.

Other important attributes that can be important but not obvious: these components are small molecules and easily reach the affected area by simple flow and molecular diffusion. They are also water miscible and readily mix with the water present in the affected area and can remain there for long periods. The ester can hydrolyze and provide lactic acid or lactate salt, which are known to be good for the skin.

The application method is very simple. After cleaning the nail area the method just requires topical application to the affected nail area by a swab or other applicator once or more times, preferably twice, per day. It is preferred that the surrounding lateral and proximal nail folds and free edge (surrounding skin tissue) also be swabbed with the biosolvent. After application, the area should not be washed for a few hours.

A contemplated biosolvent mixture can be further formulated to increase viscosity and therefore avoid wasting the composition using common thickeners (viscosifiers). Exemplary thickeners include fumed silica, bentonite and hectorite clays and so-called organophilic bentonite clays sold under the names Claytone®-40, Claytone®-AF and the like available from BYK-Chemie, GmbH, Wesel, Germany.

A particularly preferred thickener is a hydroxypropyl cellulose such as Klucel™ H, M, and G. These materials are said by their manufacturer, Ashland Inc. (Covington, Ky., USA), to have molecular weights of 1,150,000 Da, 850,000 Da and 370.00 Da, respectively, as measured by GPC-size exclusion chromatography. Of these materials, Klucel™ M is most preferred. Use of the pharmaceutical grade of this polymer is most preferred and such materials include the letter “F” in their designations.

One or more miscible fragrances, monoterpenes or monoterpene mixtures, and organic thickening or penetrating agents can be present in a biosolvent mixture. Exemplary organic thickening and/or penetrating agents utilized are a C₁-C₁₀-hydrocarbyl C₁-C₁₀-carboxylate like geranyl acetate, methyl propionate, methyl butyrate, ethyl acetate, ethyl butyrate, isoamyl acetate, pentyl butyrate, pentnyl pentoate, octyl acetate and benzyl acetate.

The word “monoterpene” is used broadly herein to include monoterpene compounds that are hydrocarbons and monoterpenoids that are oxygenated terpene hydrocarbons, and preferably hydroxyl-containing monoterpene alcohols. Illustrative monoterpenes can be obtained as purified single compounds as well as mixtures of monoterpene compounds.

Exemplary monoterpene compounds include limonene, camphor, menthol, carvone, terpineol, and thujone and mixture thereof. A monoterpene alcohol such as menthol or one of the four isomers of terpineol is particularly preferred.

It is to be understood that a commercially-obtained monoterpene product can include a plurality of monoterpenes as well as sesquiterpenes and related terpene compounds. Such mixed terpene-containing compositions are considered “a monoterpene or monoterpene mixture” if at least 50% of the mixture by weight is one or more monoterpenes or their esters.

An essential oil mixture of monoterpenes such as rosemary oil, sweet orange oil, tea tree oil (melaleuca oil), lavender oil, and peppermint oil is also contemplated for use with the alcohol and lactate ester components of a biosolvent and is considered a monoterpene mixture. Use of an oil that contains a weight percentage of about 40% monoterpene alcohol or more is preferred. It is to be understood that essential oils obtained from plants also typically contain sesquiterpenes and other terpenoid compounds, but contain more than about 40% by weight of monoterpene alcohols or their derivatives such as C₁-C₄ esters.

The word “hydrocarbyl” is used herein as a short hand term for a non-aromatic group that includes straight and branched chain aliphatic as well as alicyclic groups or radicals that contain only carbon and hydrogen. Thus, alkyl, alkenyl and alkynyl groups are contemplated, whereas aromatic hydrocarbons such as phenyl and naphthyl groups, which strictly speaking are also hydrcarbyl groups, are referred to herein as aryl groups or radicals.

No separate bioactive compound such as an antifungal compound is required for this method of removal and clearing of discoloration and debris and rendering a clear nail for a long period of time. The primary components are well known biosolvents and approved for food cosmetic and pharmaceutical applications.

Should one or more bioactive, antifungal compounds be deemed desirable to be added to a contemplated biosolvent decolorizing composition to provide an anti-fungal effect. Thus, a pharmaceutical composition for treating a dermatological fungal infection is also contemplated. Such a composition contains a known antifungal compound such as an azole-type compound or a benzoxaborole or mixture thereof included in an anti-fungal effective amount in a before-discussed biosolvent. Such a pharmaceutical composition can thus applied to a fungus-infected fingernail or toenail as discussed previously to provide an antifungal effect as well as a decolorizing effect.

These azole-type anti-fungal compounds are 5-membered aromatic ring-containing compounds whose aromatic ring contains at least one nitrogen atom and one other non-carbon atom (nitrogen, oxygen or sulfur). Most azole-type anti-fungal compounds contain two or three nitrogen atoms in their 5-membered aromatic rings (diazoles and triazoles). Those products are claimed to eliminate or abate the infection but do not clear the discolorations or debris, particularly in people that have chronic and long term infections. Imidazole, triazole and thiazole azole-type anti-fungals compounds, including those noted previously, include bifonazole, butoconazole, clotrimazole, econazole, fenticonazole, isoconazole, ketoconazole, luliconazole, miconazole, oxiconazole, sertaconazole, sulconazole, ioconazole, efinaconazole, fluconazole, isavuconazole, itraconazole, posaconazole, terconazole, and voriconazole.

Tavaborole is a boron-containing anti-fungal compound. Tavaborole and similar benzoxaborole compounds as are discussed in WO 2013/050591. A benzoxaborole that is free of ionically charged substituent groups at physiologic pH values is itself free of ionic charge at pH 7.2-7.4. Such a compound is preferred as non-charged molecules appear to penetrate the nail more readily than those with ionic charges.

An antifungal synergistic mixture of a benzoxaborole and another anti-fungal compound such as an above-described azole-type antifungal compound can be used. See, U.S. Pat. Nos. 9,737,075 and 10,085,452.

This invention is further supported by the following examples.

### Illustrative Formulations and Solubilities Therein Formulations

Two illustrative formulations were prepared and thereafter applied to the toenails of five human subjects as discussed hereinafter.

### 1) VertecBio Formulation #1 Composition

| Ingredient | Weight % |
|---|---|
| Ethyl alcohol | 78.24 |
| Ethyl lactate | 19.56 |
| Menthol | 2.00 |
| Klucel ® MF | 0.20 |
| Total | 100.00 |

### 2) VertecBio Formulation #2 Composition

| Ingredient | Weight % |
|---|---|
| Isopropyl alcohol | 68.46 |
| Ethyl lactate | 29.34 |
| Menthol | 2.00 |
| Klucel ® MF | 0.20 |
| Total | 100.00 |

In both preparations, the liquids were added together and mixed thoroughly. The Klucel® MF solid was then added and mixed with the liquid portions at a temperature of about 35° to about 40° C. until a clear, homogeneous composition was achieved. The compositions were cooled and packaged.

Solubility Studies

Study 1

Two blends of C₂-C₄ alcohol and C₂-C₄ alcohol lactate ester were prepared. The first blend contained equal weights of ethanol and ethyl lactate (50%/50% w/w). The second blend contained equal weights of isopropanol and ethyl lactate (50%/50% w/w).

20.0 Weight % tea tree oil was added to the two alcohol/ethyl lactate blends and each was mixed thoroughly at ambient temperature (about 200 to about 25° C.). Both mixtures resulted in clear, single-phase solutions.

Conclusion: Tea tree oil is readily soluble in both 50/50 blends of the two alcohols with ethyl lactate.

Study 2

Clotrimazole powder was used illustratively as an azole-type anti-fungal compound in a second solubility study. The powder was added to each of the two preformed solvent blends and the resultant composition thereafter mixed thoroughly at ambient temperature (about 20° to about 25° C.) until dissolution was complete and the composition was clear and free of particulates. The final concentration of clotrimazole in the ethyl alcohol/ethyl lactate blend was 10.31%. The final concentration of clotrimazole in the isopropyl alcohol/ethyl lactate blend was 10.07%. Both mixtures resulted in clear solutions with no visible solid particles present.

Conclusion: clotrimazole is soluble in both 50/50 blends of the two alcohols with ethyl lactate, to concentrations greater than 10% w/w.

### Example 1

A sixty-nine year old woman with a long chronic history of fungal toe infection and nail discoloration was the voluntary subject. She had been using Listerine® Original, which is sold as an antiseptic for oral use, for many months.

For this comparative test she started using a contemplated biosolvent formulation [VertecBio solvent Formulation #] as an once daily application with a cotton swab only on one (right) hallux nail and continued with the Listerine® treatment on the other (left) hallux nail.

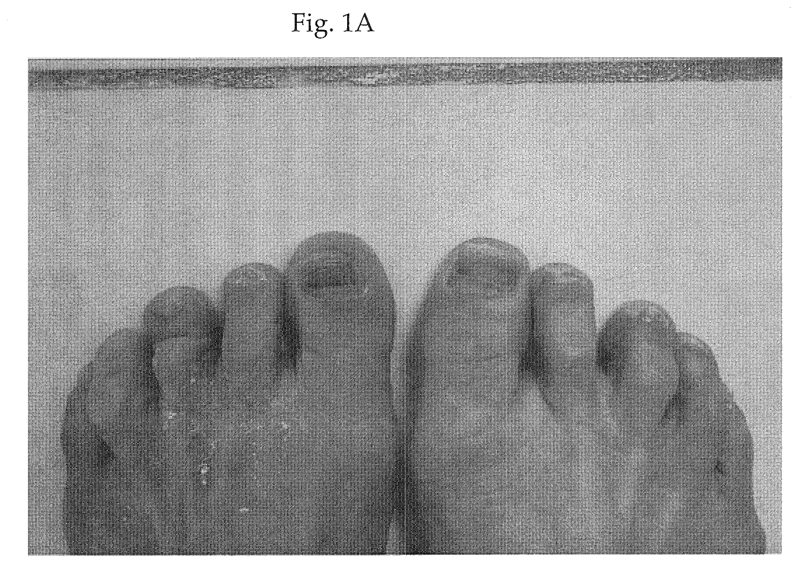

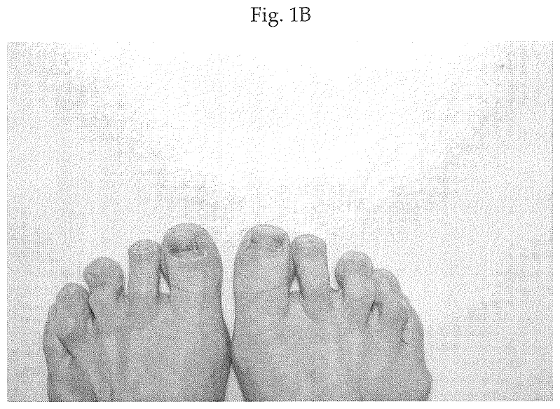

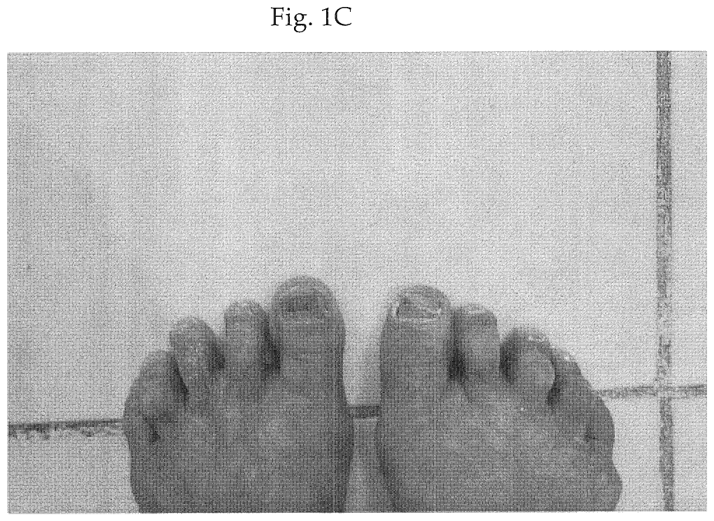

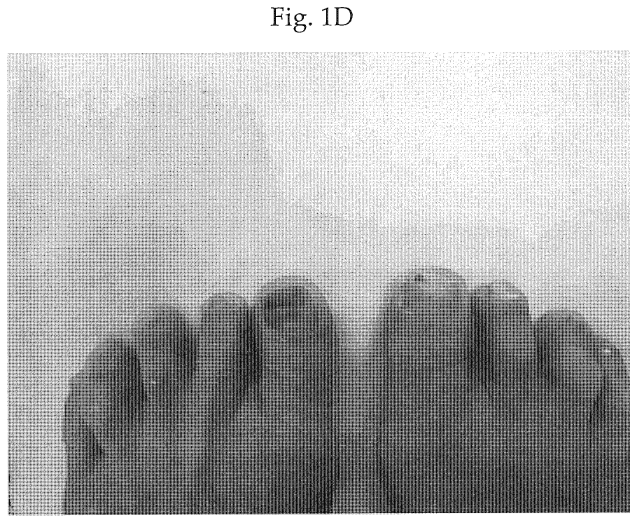

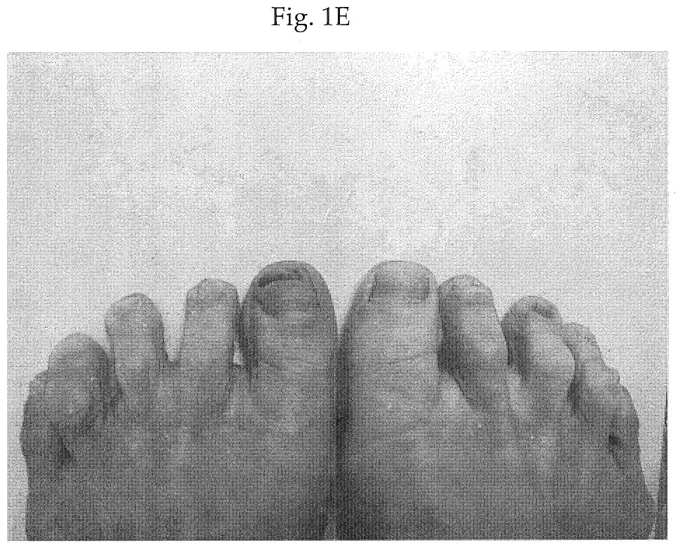

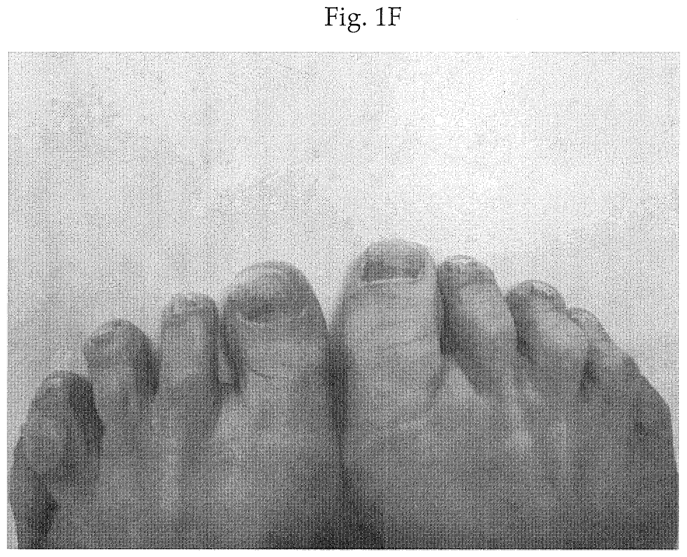

Daily observations and periodic photographs were taken to record the results. The two nails were similarly discolored at the beginning of the study. FIG. 1A is a photograph taken about 3 weeks after the beginning of the study.

The right toe kept feeling normal and had no discomfort. The left toe also had no discomfort.

After a short time period, the right toe began to feel cleaner, and right hallux nail lost its discoloration when compared to the left toenail. The photographs after about 3 weeks (FIG. 1A) and after about 5 weeks (FIG. 1B) clearly show and record these effects. FIG. 1B shows that the discoloration originally seen on the right hallux nail is not visible, and the nail and toe had returned to their normal appearance. The long accumulated biological debris was also gone. The Listerine® Original treated toenail did not show any improvement.

After about eight weeks of treatment to the right hallux nail, the left hallux nail also began to be treated with the formulation in the same manner as the right hallux and progress of both toes was monitored. After several weeks the new nail growth in the left toe appeared to be clear. A photograph taken after 8 weeks (FIG. 1D) from beginning treatment of the left hallux nail (total of 16 weeks from the beginning of the study) showed slow and continuing growth of clear nail in the left hallux. The right toe remained completely clear and healthy looking.

The treatment with the same formulation was further continued for another 16 weeks. The right toe continued to be completely clear and healthy looking. The left toe continued to look to improve and the discoloration continued to disappear and more debris continued to come out.

A photo (FIG. 1E) was taken after 16 additional weeks of application to both toenails (32 weeks total—32 weeks on right toe and 24 weeks on left toe). This photo shows the right toe looking completely normal, clear and healthy. The left hallus toenail was considerably improved and the discoloration continued to disappear. At this time the treatment on right toe was stopped and it was monitored to see if the treatment provided long term results.

The left toe treatment continued with a new formulation (VertecBio solvent Formulation #2) that contained isopropanol instead of ethanol present at the same concentration, and the same amounts of the other previously named ingredients of VertecBio solvent Formulation #1. The application treatment method remained the same as before. After another about 12 weeks, a photograph (FIG. 1F) was taken to monitor progress. As can be seen the left toe continued to clear up and the right toe remained clear and healthy looking.

The results of this long term test show:

1. Both formulations are effective.
2. The treatment provides long term results—the discolorations do not come back after the treatment is stopped.

### Example 2

A sixty-five-year-old man with a chronic toe nail fungus, primarily on the toenails of his left foot, was another volunteer test subject. The subject's condition has persisted for decades. In the past, the subject employed various topical and physical treatments that were not successful in eradicating the fungus. Topical treatments included natural substances such as tea tree oil and oil of oregano. The subject also had several laser treatments performed on the toenails of the left foot and the hallux of the right foot.

As a probable result of the laser treatment, some improvement was noted on the right hallux toenail and the second and third toenails of the left foot, but no notable change in the condition was observed on the other toenails of the left foot. However, the improved toenails had only minor fungus infection at the start of the treatment.

In 2015 the subject underwent additional laser treatment and removal of some toenails on his left foot by a podiatrist with subsequent topical treatment. The nails of the left foot hallux, fourth toe and the fifth toe were removed. The toenail of the fifth toe was also chemically treated to prevent its regrowth. The removal procedure along with laser treatment of the second and third toenails appeared to eradicate the fungus from the second, third and fourth toenails. However, even after removal, the toenail of the hallux regrew with darker coloration and the possible presence of fungus.

Eventually, full fungal attack reappeared on the left toenails of the fourth toe and to the greatest extent on the hallux. The fifth toenail also showed signs of fungal attack, but the fifth toenail, as a result of the above described chemical treatment, is extremely stunted in length and shows no further growth.

The subject began a multistep regime of physical treatment to the toenails on both feet that included: 1) keeping the toenails exposed whenever practical (i.e. remaining barefoot or wearing sandals wherever possible); 2) keeping the toenails as dry as possible; 3) use of an antibacterial scrub with a stiff brush when showering to keep the edges of the nails clean; 4) getting the toenails completely dry by using a hair dryer on the toes after showering and 5) regular exposure of the toes to sunlight. This treatment, over the course of many months, appeared to slowly yield some positive results. However, maintaining regular practice of the treatment regime was found burdensome by the subject.

The subject began treatment of only the left hallux nail, which was discolored over a long period of time, using VertecBio solvent Formulation #1 described above. The subject applied the formula to this toenail once a day on a regular basis, but occasionally missed a day (about 90 to 95% daily use consistency).

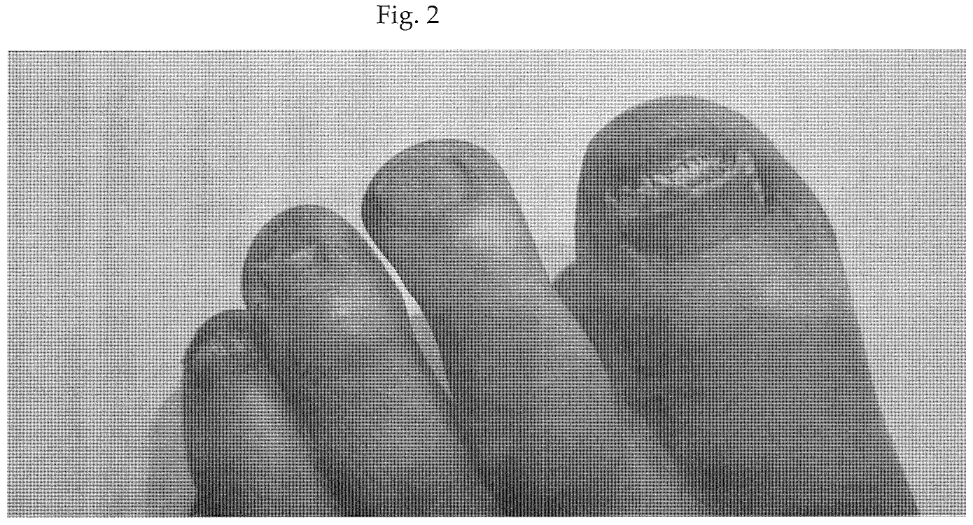

The subject also performed the above 5-step treatment sporadically on the rest of the left foot toenails. After about four weeks of treatment the lunule of the treated hallux toenail showed sign of clarity and normal growth. After about another 8 weeks, the clarity and normal growth appears to have progressed from the lunule into the nail body with the hallux nail having about a quarter inch of new growth. The condition of the toenails is described in conjunction with the following photograph shown in FIG. 2.

### Example 3

A seventy-three year old male with a chronic history of fungal toe infection and nail discoloration was a voluntary subject to test the same formulation for effectiveness in toenail discoloration. Prior treatment of the large toe (hallux) and second toe on his right foot with Ciclopirox Olamine Cream USP, 0.77% had proved ineffective. For this study, he started using VertecBio solvent Formulation #1 (once daily application with a cotton swab) on the 2 subject toes. The 2 toes felt normal and had no discomfort from the application.

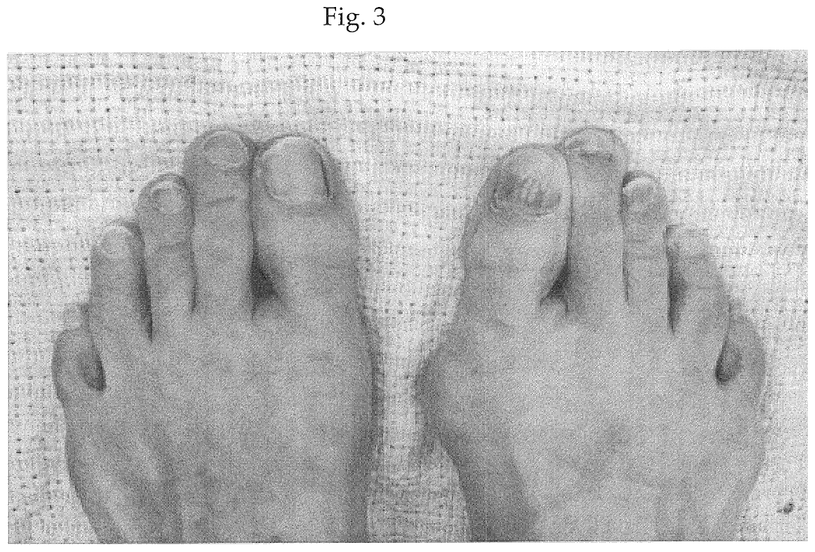

After several weeks, the discoloration of the toes began to slowly lessen. After 10 weeks of application, the appearance of the 2 toes was noticeably improved. After 6 months, the feel and appearance of the old growth on the 2 toenails was stable and new, clear growth was slowly becoming apparent. FIG. 3 shows the subject's feet six months after treatment of the hallux and second nails of the right foot had begun.

### Example 4

Subject 4 is a 78 year old male who has suffered from a persistent toe fungus problem and discoloration for the last 30 years. His left toe was highly discolored with brown and green streaks that were highly unsightly. Various topical creams and ointments were prescribed by his doctors and were used, but none of them produced any visible results. Tests by dermatologists showed no active fungi but the discolorations did not go away.

The subject was going to try a new laser procedure but elected to experiment with the VertecBio solvent Formulation #1. He applied this product twice a day using a cotton swab for a period of approximately 20 weeks. Signs of clearing of the discolorations began after about 12 weeks.

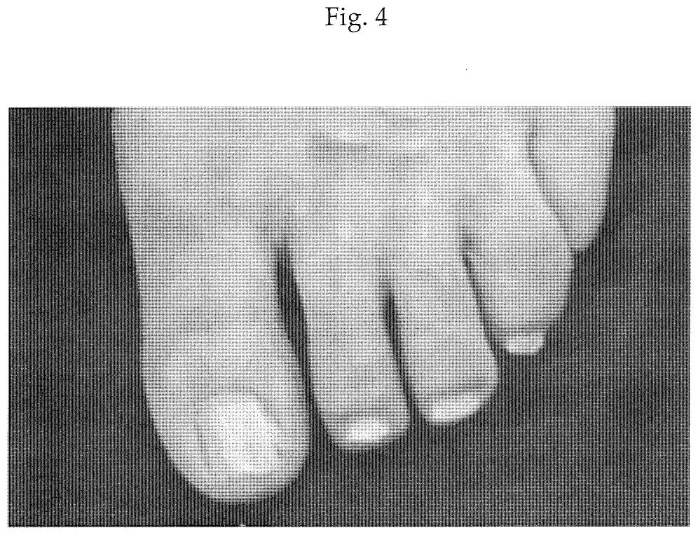

He switched to VertecBio solvent Formulation #2 using the same application method or an additional 12 weeks. As shown in the photograph (FIG. 4), the results have proven to be highly encouraging. The color of the left toe has returned to near normal and there are no visible stains. The subject continued to use these formulations to ensure that the discolorations do not reappear.

This long term test establishes:

1. Both formulations are effective.
2. The treatment clears up discolorations that remain long after any fungal infection has been cured.

### Example 5

A seventy-six-year-old man with a chronic toe nail fungus was another volunteer test subject (Subject 5). The subject's condition had persisted for more than a decade. No medicinal or other treatments were previously attempted to treat the condition.

Several months prior to beginning treatments, the right hallus nail fell off. Regrowth of the nail and its characteristic discoloration followed.

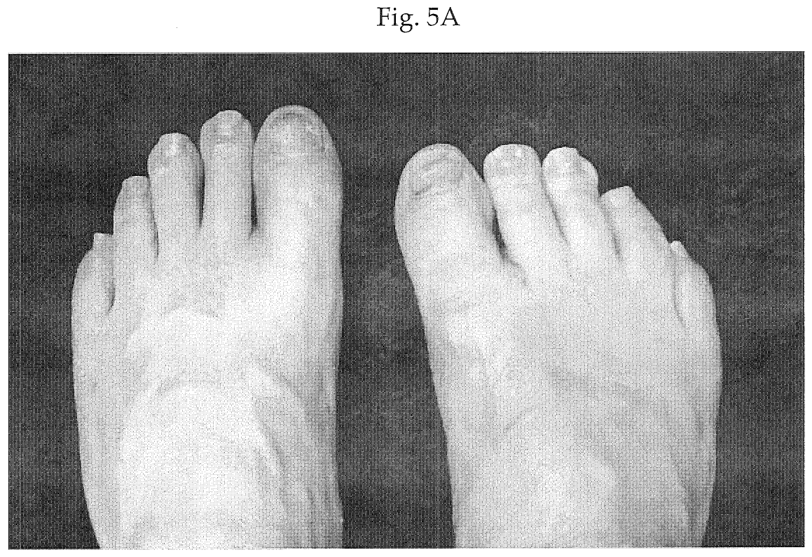

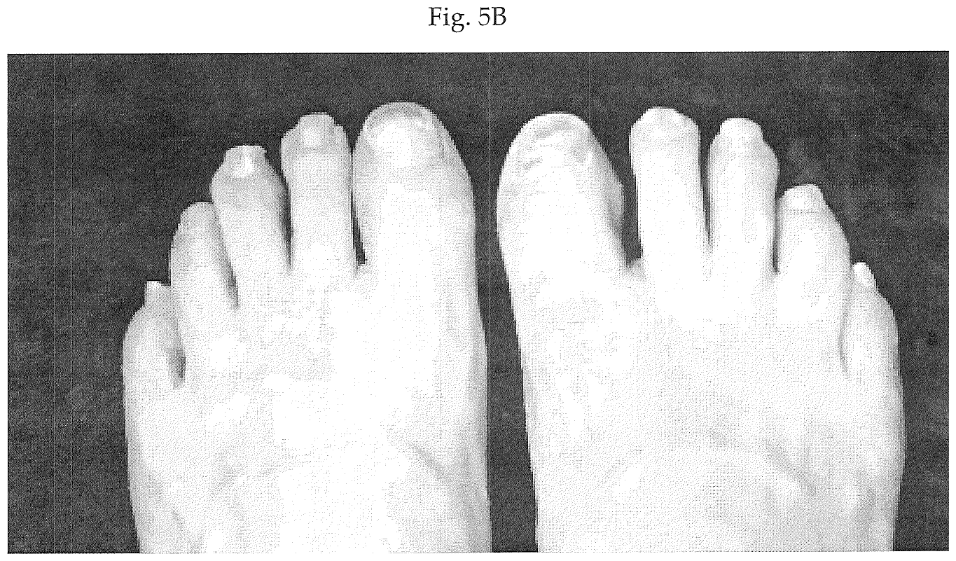

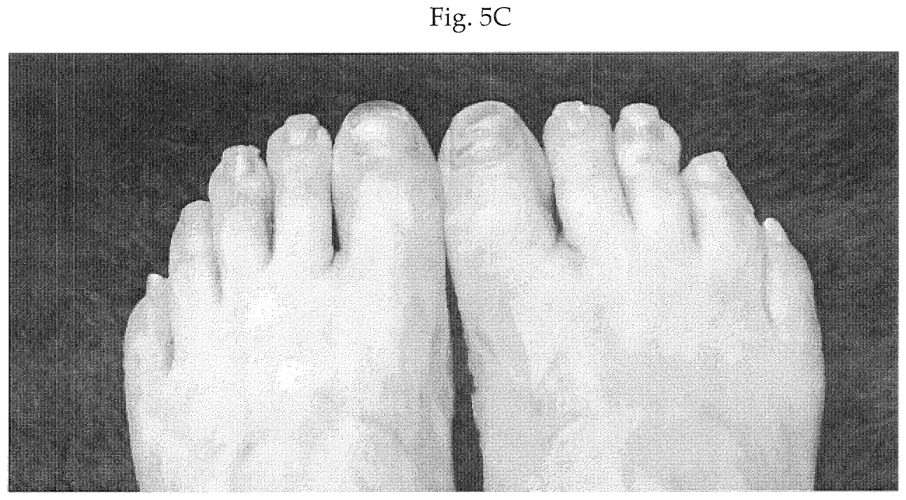

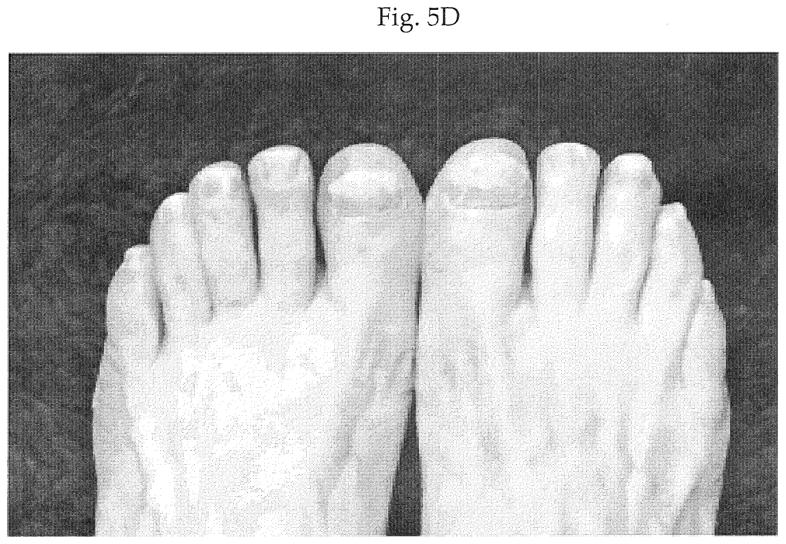

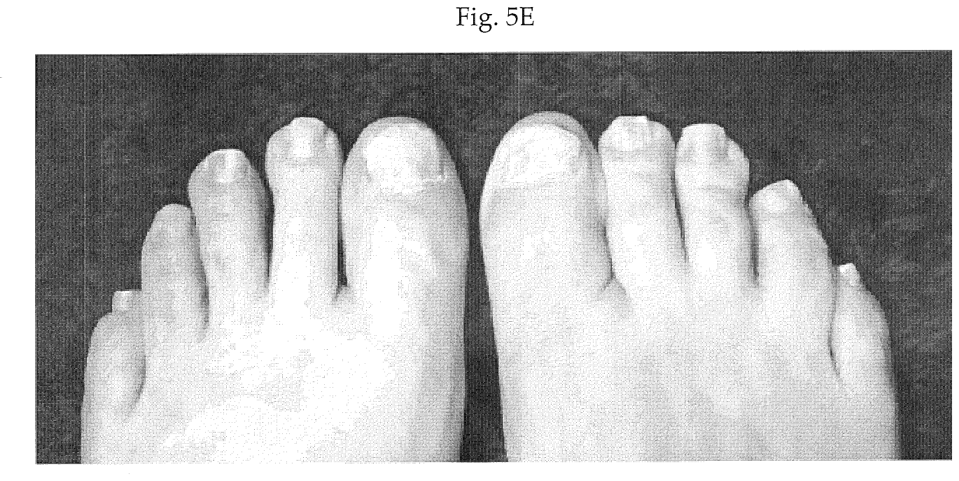

The subject began application of VertecBio solvent Formulation #1 described earlier to all of the nails of both feet. The subject applied the formula to this toenails once a day on a regular basis. Photos of the subject's feet and toenails were taken prior to the beginning of the study (FIG. 5A) and approximately monthly thereafter (FIGS. 5B-5E).

After about four weeks beginning applications, the lunules of the treated hallux toenails showed signs of clarity and normal growth (FIG. 5B). After about another 3 weeks, the clarity and normal growth appear to have progressed from the lunules into the nail body with the hallux nails having about a quarter inch of new growth (FIG. 5C). The subject also trimmed his nails two days after the photo of FIG. 5C was taken.

About seven weeks after the start of any biosolvent formulation application to the subject's toenails, the subject switched to administration of VertecBio solvent Formulation #2 as discussed before. The first photo after the change to administration of VertecBio solvent Formulation #2 was taken about six weeks after the change to VertecBio solvent Formulation #2 was made (FIG. 5D).

FIG. 5D shows the continued toenail growth about six weeks after that shown in FIG. 5C. It is seen that the horizontal brown bands traversing the right hallus nail perpendicular to the direction of growth have grown out and have not been replaced. The photo of FIG. 5E, taken about one month after that of FIG. 5D, shows that substantially all of the brown-colored material beneath the toenails has gone and has not been replaced.

As shown most readily in FIG. 5E, the tissue beneath the hallux toenails from the proximal nail fold to about 40% of the way to the free edge looked normal and pink, whereas the remaining about 60% of that tissue was obscured by the discolored, yellowish nail.

The results from Subject 5 also show that both formulations are effective. The Subject noted apparent normal growth after about four weeks of once-daily applications. Both of VertecBio solvent Formulation #1 and #2 appeared to work equally well.

Each of the patents, patent applications and articles cited herein is incorporated by reference. The articles “a” and “an” are used herein to refer to one or to more than one (i.e., to at least one) of the grammatical object of the article. By way of example, “an element” means one element or more than one element.

The foregoing description and the examples are intended as illustrative and are not to be taken as limiting. Still other variations within the spirit and scope of this invention are possible and will readily present themselves to those skilled in the art.

1. A method for removing infection-caused nail discoloration that comprises the steps of
a) contacting a fingernail and/or toenail in need with a composition comprising a mixture of two major components, A and B, wherein Component A is a C₂-C₄ alcohol that is present at about 90% to about 20% (w/w), and Component B is a lactate ester of the same or different C₂-C₄ alcohol(s) that is present at about 10% to about 80% (w/w), said composition optionally containing up to about 5% (w/w) each of one or more of (i) a monoterpene or monoterpene mixture, (ii) thickening agent and (iii) C₁-C₄ alcohol C₁₂-C₂₀ carboxylate;
b) maintaining said contact in the absence of water contact for a time period sufficient for the composition to be sorbed by the nail and/or evaporate, and
c) repeating steps a) and b) at least 5 to about 7 times per week until said nail discoloration is no longer observable.

2. The method for removing infection-caused nail discoloration according to claim 1, wherein said C₂-C₄ lactate ester is present at about 15% to about 40% (w/w), and said C₂-C₄ alcohol is present in an amount of about of 85% to about 60% (w/w).

3. The method for removing infection-caused nail discoloration according to claim 1, wherein said C₂-C₄ lactate ester is ethyl lactate.

4. The method for removing infection-caused nail discoloration according to claim 1, wherein said C₂-C₄ alcohol is ethanol or isopropanol.

5. The method for removing infection-caused nail discoloration according to claim 1, wherein said C₂-C₄ lactate ester is ethyl lactate and said C₂-C₄ alcohol is ethanol.

6. The method for removing infection-caused nail discoloration according to claim 1, wherein said composition is free of added water.

7. The method for removing infection-caused nail discoloration according to claim 1, wherein the lateral and proximal nail folds and free edge that surround a contacted nail are also contacted with said composition.

8. The method for removing infection-caused nail discoloration according to claim 1, wherein said composition further includes about 0.1 to about 5% (w/w) of a monoterpene or monoterpene mixture.

9. The method for removing infection-caused nail discoloration according to claim 8, wherein said monoterpene is menthol.

10. The method for removing infection-caused nail discoloration according to claim 1, wherein said steps a) and b) are repeated twice daily.

11. A pharmaceutical composition for treating a dermatological fungal infection comprising a composition containing an antifungal effective amount of an antifungal compound dissolved or dispersed in a liquid composition comprising a mixture of two major components, A and B, wherein Component A is a C₂-C₄ alcohol that is present at about 90% to about 20% (w/w), and Component B is a lactate ester of the same or different C₂-C₄ alcohol(s) that is present at about 10% to about 80% (w/w), said pharmaceutical composition optionally containing up to about 5% (w/w) each of one or more of (i) a monoterpene or monoterpene mixture, (ii) a thickening agent and (iii) a C₁-C₄ alcohol C₁₂-C₂₀ carboxylate.

12. The pharmaceutical composition according to claim 11, wherein said C₂-C₄ lactate ester is present at about 15% to about 40% (w/w), and said C₂-C₄ alcohol is present in an amount of about of 85% to about 60% (w/w).

13. The pharmaceutical composition according to claim 11, wherein, said C₂-C₄ lactate ester is ethyl lactate.

14. The pharmaceutical composition according to claim 11, wherein said C₂-C₄ alcohol is ethanol or isopropanol.

15. The pharmaceutical composition according to claim 11, wherein said C₂-C₄ lactate ester is ethyl lactate and said C₂-C₄ alcohol is ethanol.

16. The pharmaceutical composition according to claim 11, wherein said composition is free of added water.

17. The pharmaceutical composition according to claim 11, wherein said antifungal compound is an azole-type fungicide.

18. The pharmaceutical composition according to claim 17, wherein said azole-type fungicide is selected from the group consisting of one or more of bifonazole, butoconazole, clotrimazole, econazole, fenticonazole, isoconazole, ketoconazole, luliconazole, miconazole, oxiconazole, sertaconazole, sulconazole, ioconazole, efinaconazole, fluconazole, isavuconazole, itraconazole, posaconazole, terconazole, and voriconazole.

19. The pharmaceutical composition according to claim 11, wherein said antifungal compound is a benzoxaborole compound that is free of ionic charge at physiological pH values.

20. The pharmaceutical composition according to claim 19, wherein said benzoxaborole compound that is free of ionic charge at physiological pH values is tavaborole.
